(19) United States
(12) Patent Application Publication
(10) Pub. No.: US 2011/0301450 A1
(43) Pub. Date: Dec. 8, 2011

(54) MAGNETIC RESONANCE IMAGING MEDIATED RADIOFREQUENCY ABLATION

(76) Inventors: Yik-Kiong Hue, College Park, MD (US); Abraham Roth, Kefar Hasiddim (IL); Jerome Leonard Ackerman, Newton, MA (US); Erez Nevo, Baltimore, MD (US)

(21) Appl. No.: 13/098,971

(22) Filed: May 2, 2011

Related U.S. Application Data
(60) Provisional application No. 61/343,591, filed on Apr. 30, 2010.

(51) Int. Cl. A61B 5/055; A61B 6/00

(52) U.S. Cl. 600/411

(57) ABSTRACT

Radiofrequency ablation (RFA) may be used as a minimally invasive treatment of solid tumors, typically cancers of the liver, lung, breast, kidney and bone, most often via a percutaneous approach. In RFA tumor tissue is killed by heating. RFA requires guidance using an imaging method to correctly position the RF applicator. Magnetic resonance imaging (MRI) can be used for guidance, and offers the additional advantage of the ability to image tissue temperature. Because MRI employs high power RF fields, the MRI scanner could serve as the source of RF energy for ablation. Described herein are an MRI-driven RF ablation device and method. The device has minimal electrical circuitry, and uses the MR scanner radio frequency field as the energy source to generate heat in tissue using an antenna and a needle. Based on the Faraday induction law, different embodiments for coupling the body coil RF energy into tissue are disclosed.

### CROSS-REFERENCE TO RELATED APPLICATIONS

This application claims priority benefit from U.S. Provisional Patent Application No. 61/343,591, filed Apr. 30, 2010, the contents of which is incorporated herein by reference in its entirety.

### FIELD OF THE INVENTION

The present invention relates to radiofrequency ablation using a magnetic resonance imaging scanner, and, more particularly to a system and method for performing radiofrequency ablation of tissue using minimal electrical circuitry and using a magnetic resonance scanner radiofrequency field as an energy source.

### BACKGROUND OF THE INVENTION

A variety of thermal tissue ablation techniques, including radiofrequency, microwave, laser and focused ultrasound, have been described which cause cell death by coagulation necrosis and/or apoptosis. The techniques involve heating the tissue above 60° C., leading to protein denaturation and membrane breakdown, and resulting in irreversible thermal damage. Among these techniques, percutaneous radiofrequency ablation (RFA), introduced almost two decades ago for the treatment of osteoid osteomas and later for primary and metastatic liver tumors, gained attention because it is effective, safe, minimally invasive, low in cost and far less traumatic to the patient compared to surgery, chemotherapy or radiation therapy. Its application has been expanded to many other cancers.

An imaging modality, e.g., ultrasound (US), contrast-enhanced computed tomography (CT) or magnetic resonance imaging (MRI), is required to guide the placement of the RFA needle (RF applicator); US and CT are the most commonly used modalities in RFA procedures. US guidance is inexpensive and rapid because of its inherent real-time capability, but it has poor image quality. Additionally, gas bubbles sometimes produced by tissue vaporization during heating limit the utility of US to monitor the treatment. Multiple sessions are required for RFA treatment under US guidance. CT is capable of multi-planar imaging, but its poor soft tissue contrast requires the administration of an exogenous contrast agent to provide clear delineation of tumor tissue and may not permit visualization of induced coagulation. Ionizing radiation exposure of both the patient and the physician further detracts from the benefits of x-ray guidance. MRI exhibits high soft tissue contrast, and is capable of imaging tissue temperature and other thermal effects. There are fast MR imaging methods allowing near real-time monitoring of the treatment. These benefits of MRI guidance are offset by the requirement for RFA equipment which is compatible with the strong static, gradient and RF magnetic fields of the scanner, and which does not introduce noise or distortion into the images, as well as the expense of extended periods of MR scanner usage for treatment. However, with the increasing popularity of interventional MR systems, MR-guided RFA has the potential to grow dramatically in use.

There have been few reports of near real-time RFA monitoring using MR thermometry because the RFA generator can create electrical interference with MR image acquisition. The generator must be placed at a safe distance from the scanner and connected to an MR-compatible RF applicator using MR-compatible cable. Alternating between MR imaging and application of heat to prevent image artifacts would defeat the usefulness of MR thermometry because heat would be carried away by tissue perfusion and the tumor temperature would drop during the transition between imaging and heating.

Patents relating to use of MRI-guided ablation with external sources of RF energy include, for example, U.S. Pat. No. 6,701,176 to Halperin et al.; U.S. Pat. No. 6,904,307 to Karmarkar et al.; and U.S. Pat. No. 7,155,271 to Halperin et al.

A combined imaging (MRI) and heating (with RF energy) device is disclosed by Kandarpa et al. (U.S. Pat. No. 5,323,778). However, the heating device disclosed therein grounds the tissue eddy currents which are produced by the alteration of the magnetic field resulting from activation of the MRI radio frequency source. Thus the probe must be grounded, for example to the hardware of the scanner. The coupling to the scanner complicates the hardware of the device, increases the risk to the scanned subject and greatly complicates the regulatory process as it must be done as an integral component of the scanner. Additionally, the device disclosed therein requires the use of a tuned coil at its tip to serve as a receiver RF coil connected to the MRI scanner to enable the determination of its position within the patient.

Thus, there is a need for a device which does not require that the device act as a receiver RF coil and which does not use an RF coil at its tip, which does not require a separate ground, and wherein heating may be controlled mechanically in addition to electronically.

### SUMMARY OF THE INVENTION

The present invention discloses a wireless device that harvests energy from the RF transmission of the scanner and has no conductive connection to another system.

There is provided, in accordance with embodiments of the present invention, a wireless heat ablation device for use inside a bore of a magnetic resonance imaging scanner. The device includes an antenna configured to wirelessly receive RF energy from the magnetic resonance imaging scanner, and a probe having an electrically conductive tip, electronically connected to the antenna, configured to receive the RF energy, and further configured to be positioned within tissue and to provide heat to the tissue by the RF energy.

In further features of the present invention, the device may also include a control unit in electrical communication with the antenna, configured for receiving the RF energy from the antenna, for coupling the received RF energy, and for sending the coupled RF energy to the probe. In some embodiments, the control unit may include an impedance matching device. In some embodiments, the control unit may include a tuning circuit. The tuning circuit may include a variable capacitor and a variable inductor, and may be configured to vary a length of the antenna. In some embodiments, the antenna has a loop circuit. The dimensions of the loop circuit may be, for example, 10-50 cm in length and width. In other embodiments, the antenna may be made of a wire. The length of the wire may be, for example, 20-80% of a wavelength of the RF signal, and the wire may in some embodiments be made of rods and joints such that the wire may be folded or expanded. In some embodiments, the electrically conductive tip is an un-insulated distal portion of a length of an insulated electrically conductive needle, hollow tube, or catheter.

There is provided, in accordance with additional embodiments of the present invention, a method of heat ablation for use inside the bore of a magnetic resonance imaging scanner. The method includes positioning a wireless heat ablation device inside the bore of the magnetic resonance imaging scanner, the magnetic resonance imaging scanner providing RF transmission at a frequency, tuning the wireless heat ablation device to approximately the frequency of the RF transmission of the scanner, receiving RF energy in the wireless heat ablation device based on the RF transmission, providing heat to a tip of the wireless heat ablation device based on the received energy, determining a treatment location for the heat ablation device by imaging a treatment area using the magnetic resonance imaging scanner, and heating the treatment location using the heated tip of the wireless heat ablation device.

In accordance with further features of the present invention, the heat may be controlled by changing the tuning of the wireless heat ablation device, by changing the average power of radiofrequency transmission of the magnetic resonance imaging scanner, or by some combination thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The above and further advantages of the present invention may be better understood by referring to the following description in conjunction with the accompanying drawings in which:

FIGS. 1A and 1B are schematic and block diagram illustrations of an MRI scanner 12 which can be used in accordance with embodiments of the present invention;

FIG. 2 is a schematic illustration of an ablation device in accordance with embodiments of the present invention;

FIG. 3 is a partially schematic and partially block diagram illustration of the device of FIG. 2, in accordance with embodiments of the present invention;

FIG. 4 is a schematic illustration of a needle from the device of FIG. 2, in accordance with embodiments of the present invention;

FIG. 5A is a circuit diagram showing an antenna of the device of FIG. 2 having a pickup loop circuit configuration;

FIG. 5B is a schematic illustration of the pickup loop circuit configuration of FIG. 5A;

FIG. 6 is a schematic illustration of the pickup loop circuit configuration of FIGS. 5A and 5B as positioned within an MRI scanner;

FIG. 7A is a diagram showing an antenna of the device of FIG. 2 having a wire configuration;

FIG. 7B is a circuit diagram in accordance with the wire configuration of FIG. 7A;

FIG. 7C is a schematic illustration of the wire configuration of FIGS. 7A and 7B;

FIG. 8 is a schematic illustration of the wire configuration of FIGS. 7A-7C as positioned within an MRI scanner;

FIG. 9 is a schematic illustration of the antenna of FIGS. 7A-7C and 8 having a wire configuration, shown in an expanded state;

FIG. 10 is a schematic illustration of the antenna of FIGS. 7A-7C and 8 having a wire configuration, shown in a folded state;

FIGS. 11A and 11B are schematic illustrations of tuning circuits, in accordance with embodiments of the present invention;

FIG. 11C is a schematic illustration of an impedance matching device, in accordance with embodiments of the present invention;

FIG. 12 is a graphical illustration showing the temperature at the tip of a coaxial cable as a function of time for square loop circuits;

FIG. 13 is a graphical illustration showing the temperature measured at the tip of different length wires as a function of heating time;

FIG. 14 is a graphical illustration showing a comparison of a wire with a square loop; and

FIG. 15 is a graphical illustration showing thermal imaging results in a specimen of bovine liver using a long wire pickup antenna.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF THE INVENTION

In the following detailed description, numerous specific details are set forth in order to provide a thorough understanding of the present invention. It will be understood by those of ordinary skill in the art that the present invention may be practiced without these specific details. In other instances, well-known methods, procedures, components and structures may not have been described in detail so as not to obscure the present invention.

Before explaining at least one embodiment of the present invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

MRI scanners are equipped with RF generators capable of many kilowatts of peak RF power output, and this RF power can be precisely controlled by the pulse sequence. Most of the RF power applied to the body coil of the scanner is not dissipated in the patient, but rather in the coil itself. To prevent excessive general tissue heating, specific absorption rate (SAR) monitoring is incorporated into every clinical MRI scanner. However, the overall spatial distribution of RF power dissipation in the subject may be altered by conductive structures placed within the RF coil so as to create local “hot spots” in tissue. For example, the potential of RF burns from improperly routed cables, metallic jewelry, implanted devices, EKG leads, etc., is well known. By harnessing this effect to intentionally create zones of tissue heating, we can achieve the goals of RFA by means of the scanner and passive conductive devices alone, while gaining all the benefits of intraprocedural MRI to guide and monitor the treatment.

The proposed invention for MRI-mediated radiofrequency ablation uses Faraday induction to couple RF energy from the body coil of the scanner to an RF energy capture device, which then conducts the RF energy to the treatment zone. This device can be as simple and inexpensive as a wire appropriately routed on the patient table, and terminating in a needle inserted into the tumor. The effectiveness of the device depends on its geometry and its electrical network properties, as well as the Larmor frequency and scanner coil geometry.

In the present invention a novel radiofrequency (RF) ablation device for use in magnetic resonance imaging scanners is introduced which does not require an external RF power generator or connections to any external system. This ablation device has minimal circuitry and does not require a grounding pad to complete the electrical path. This eliminates the possibility of accidental skin burns due to poor contact of the grounding pad. In effect, the capacitance of the patient's body with respect to the surroundings forms the ground path.

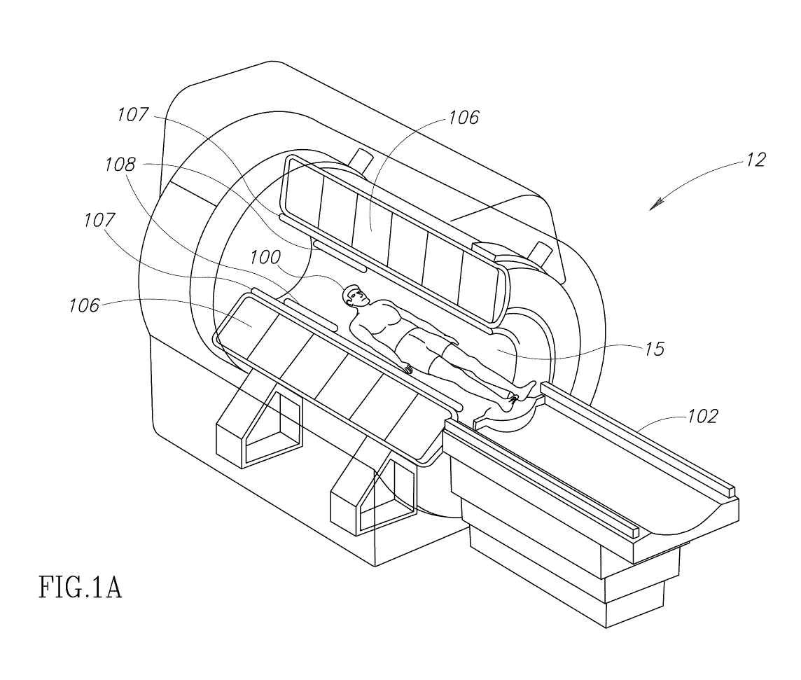

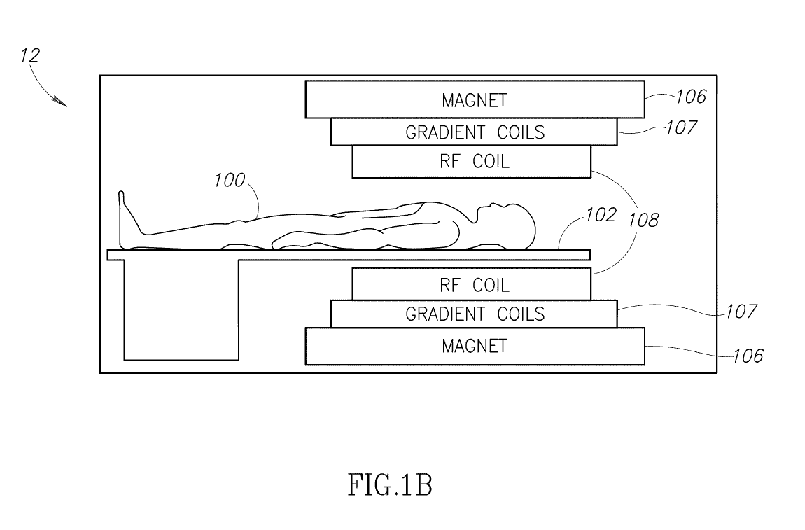

Reference is now made to FIGS. 1A and 1B, which are schematic and block diagram illustrations of an MRI scanner 12 which can be used in accordance with embodiments of the present invention. MRI scanner 12 includes static magnetic coils 106 and gradient magnetic coils 107. MRI scanner 12 further includes an embedded body RF coil 108. The inner surface of RF coil 108 is covered with a bore tube 15 to enclose RF coil 108 and to protect a patient 100 from contact with it. Patient 100 may be positioned on a patient table 102 and placed inside bore tube 15. RF coil 108 is capable of generating a spatially homogeneous RF field. In most MRI scans, RF coil 108 is used to transmit RF power to excite nuclear spins within patient 100. RF coil 108 is usually of a birdcage design. When properly tuned to the correct electromagnetic mode, RF power applied to a first port of RF coil 108 will excite a homogeneous linearly polarized magnetic field within RF coil 108 and is naturally decoupled from a second port which is geometrically rotated 90° away from the first port. Driving both first and second ports with RF power in phase quadrature excites a circularly polarized (CP), or rotating, magnetic field, which is more effective in exciting nuclear spins than a linearly polarized field. Either the same RF coil 108 or a separate RF coil (not shown) detects the precessing nuclear magnetization which constitutes the signal from which images are reconstructed. Other geometric configurations of MRI scanners, magnetic coils and RF coils are possible and may be used effectively with the invention.

In the present invention, patient 100 is positionable inside bore 15 of MRI scanner 12, and a radiofrequency (RF) ablation device 16 is provided within bore 15 of MRI scanner 12, for accessing a lesion within patient 100. RF ablation device 16 is configured to receive RF energy from RF coil 108 via an antenna or other RF pickup device, and is further configured to use the RF energy to heat the lesion.

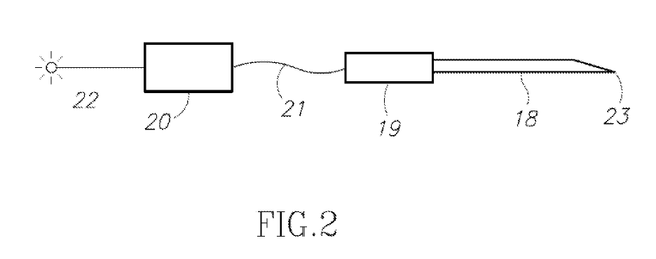

Reference is now made to FIG. 2, which is a schematic illustration of device 16 in accordance with embodiments of the present invention. Device 16 includes a probe 18 for accessing the lesion within patient 100. Access is made directly through the skin and into the lesion. As such, probe 18 is generally comprised of a needle tip 23. Probe 18 is attached to a handle 19, which is configured to be held by a user applying the RF ablation treatment to patient 100. A connecting cable 21 connects handle 19 and probe 18 to a control unit 20. Connecting cable 21 is configured to both mechanically and electrically connect probe 18 to control unit 20, generally through handle 19. An antenna 22 is in electrical communication with control unit 20, and is configured to receive RF energy from MRI scanner 12, as will be described in greater detail hereinbelow.

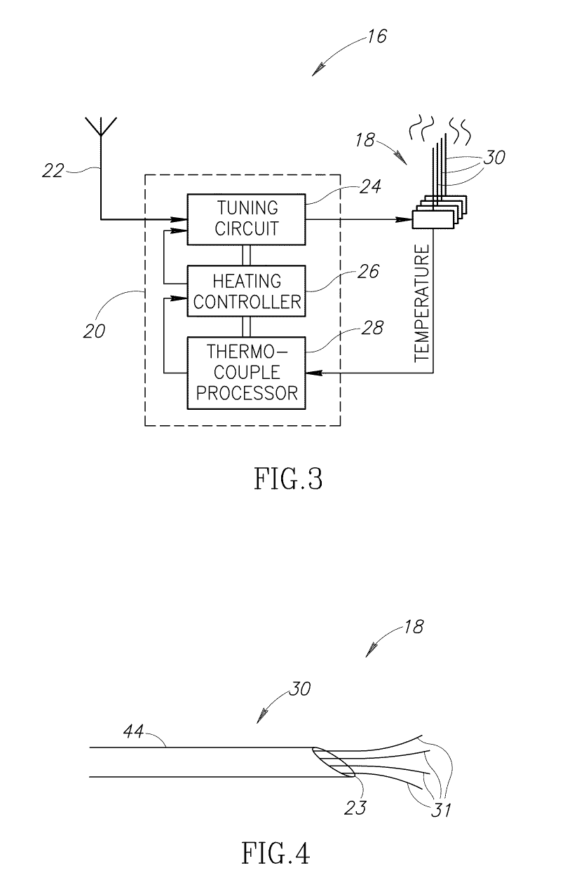

Reference is now made to FIG. 3, which is a partially schematic and partially block diagram illustration of device 16, in accordance with embodiments of the present invention. Antenna 22 is in electrical communication with probe 30 via control unit 20. Control unit 20 may include one or multiple components. In the embodiment shown herein, control unit 20 includes a tuning circuit 24, a heating controller 26, and a thermocouple processor 28. Tuning circuit 24 receives RF energy via antenna 22, and after proper tuning, couples the RF energy to probe 18 to produce heat. Probe 18 includes one or multiple needles 30, which are configured to provide heat to the tissue being treated. In some embodiments, the temperature of the heat emitted via needles 30 is measured via a temperature sensor, and this information is sent back to control unit 20. In the configuration shown in FIG. 3, a designated thermocouple processor 28 is configured to receive information about the temperature, and to send this information to a heating controller 26. Heating controller 26 then sends the information to tuning circuit 24, which can then adjust the signal sent to probe 18 to either increase or decrease heat depending on the temperature measurements. It should be readily apparent that in some embodiments, a thermocouple processor 28 is not used, and tissue temperature information may be measured from images generated by MRI scanner 12. Additionally, in some embodiments a heating controller 26 is not used, and heating control is accomplished by varying the RF power delivered by the MRI scanner. The temperature can then be adjusted either via a control unit within MRI scanner 12, or this information is sent to control unit 20, which can then adjust the RF energy coupled to needles 30 accordingly. It should also be readily apparent that instead of a thermocouple to sense temperature, other types of sensors, including but not limited to thermistors, resistance temperature devices (RTDs) and fiber optic fluoroscopic sensors, may be used. In some embodiments, control unit 20 includes an impedance matching device instead of a tuning circuit, as will be explained further hereinbelow.

Reference is now made to FIG. 4, which is a schematic illustration of one of needles 30, in accordance with embodiments of the present invention. Probe 18 includes one or multiple needles 30, each of which may contain one or more electrodes 31. Electrodes 31 may be housed in a sleeve 44. In some embodiments, a distal end of sleeve 44 is needle tip 23. Electrodes may be retractable into sleeve 44 during puncture through the skin of patient 100 via needle tip 23, and may then be extended to the lesion and used to apply heat. In some embodiments, multiple needles 30 may be placed in various locations, such as, for example, different tumors to enable treatment of a larger volume of tissue.

In embodiments of the present invention, the law of electromagnetic induction is employed by placing a linear or loop electrical conductor (i.e., antenna 22) in the rotating RF magnetic field of RF coil 108. By doing so, an electromotive force (EMF) is induced in antenna 22 by Faraday's law of induction, in precise analogy to an electric power generator in which an EMF is induced in a wire loop rotating in a static magnetic field. Although transformer induction and motional induction are discussed in the next two sections as distinct phenomena leading to two separate embodiments of the ablation devices, they are two complementary aspects of the single law of electromagnetic induction.

### Faraday's Law of Transformer Induction

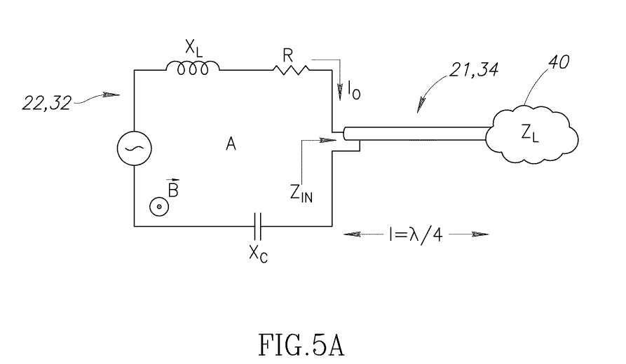

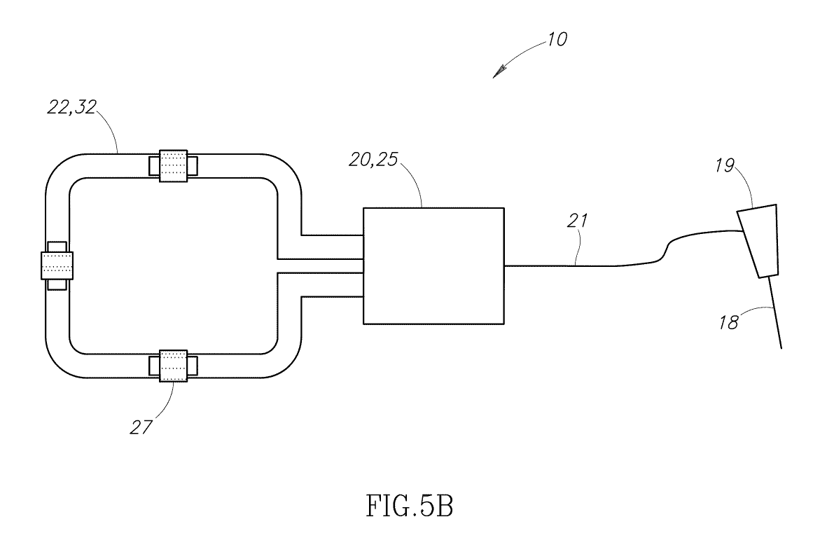

Faraday's law of transformer induction states that a changing magnetic flux through a fixed conductive RF pickup loop induces an EMF around the loop. In one embodiment shown in FIG. 5A, antenna 22 has a pickup loop circuit configuration. In FIG. 5A, antenna 22 is connected to connecting cable 21, which is a quarter wavelength transmission line 34 acting as an RF applicator, and may also serve as a needle 30, with its center conductor serving as an electrode 31. The circuit terminates in a tissue volume 40 with effective impedance Z_{L}. Although the inductance L of the loop is a characteristic of the entire physical geometry of the loop, it is represented in the circuit diagram as a lumped inductance L with inductive reactance X_{L}. The lumped resistance R represents all of the circuit losses of the loop. The capacitance C, introducing capacitive reactance X_{C} into the circuit, serves to resonate the loop at the scanner frequency, or to reduce or minimize the total loop reactance. In some embodiments, variations may be used which do not include the capacitor. In other embodiments, multiple capacitors in series may be used. The pickup loop is placed within RF coil 108 such that the loop axis aligns with a component of the oscillating magnetic flux density vector B (designated by the dotted circle indicating the component of vector B coming out of the plane of the loop). An alternating current flows within the loop driven by an electromotive force (EMF) described by the transformer induction law. Ohmic heating (via current flowing through the tissue) and dielectric heating (via the loss of motion of molecular dipoles induced by the RF potential) occur primarily in the region of the tip of the RF applicator and to some extent along the needle length. Since the magnetic flux is a periodic function of time, the current within the loop I₀ of area A and the current within the tissue I_{L} can be represented in phasor form as

I₀=ωAB/((X_{L}−X_{C})+j(R+Zᵢₙ)) (1)

I_{L}≈I₀(1−Γ_{L}) (2)

where ω is the angular frequency of MRI scanner 12, B is the magnitude of the component of the magnetic flux density parallel to the loop axis, X_{L} is the inductive reactance of the loop, R is the resistance, X_{C} is the capacitive reactance of a capacitor used to tune the loop, Zᵢₙ is the input impedance of the transmission line and Γ_{L} is the reflection coefficient due to the impedance mismatch between the RF applicator and the tissue. The approximation symbol in Equation 2 takes account of the fact that the transmission line may be lossy, and that these usually small losses are disregarded in this analysis. Including the losses complicates the analysis but does not affect the invention. To maximize the current flow, and therefore the heating, in the tissue, a variable capacitor may be used to tune the loop to the scanner operating frequency so that the loop reactance is minimized. In addition, a better impedance match between the tissue and the transmission line would increase the heating efficiency further. However, to keep the mechanical structure of the RF applicator probe simple, no impedance matching is included at the tip in this embodiment of the invention, although it could be included and would be within the scope of the invention. It should be clear that the pickup loop of the present invention is intended to couple to the body RF coil of the scanner, rather than to the nuclear spins.

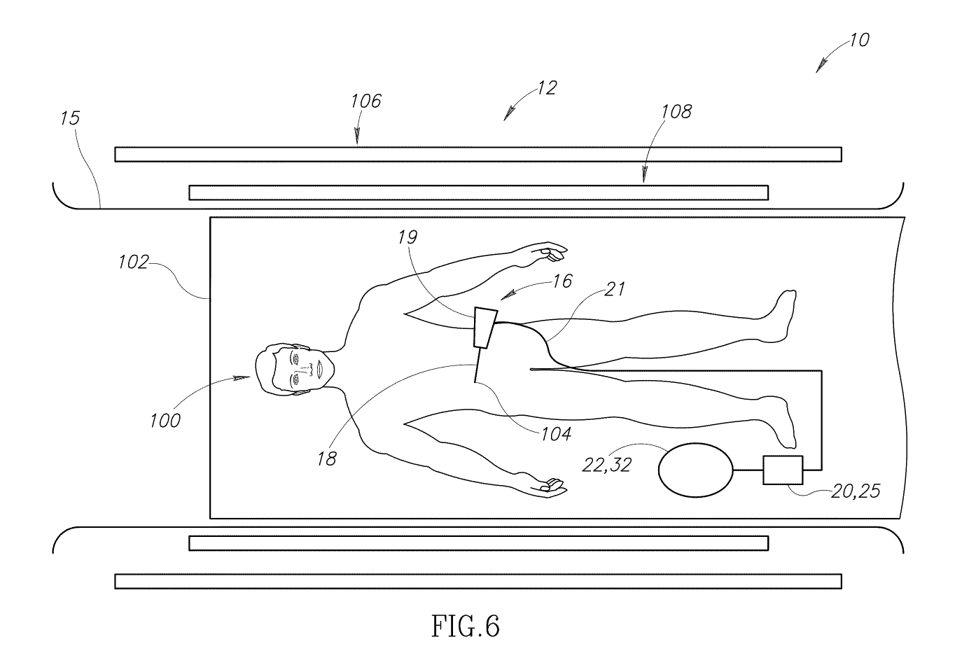

Reference is now made to FIGS. 5B and 6, which are schematic illustrations of system 10 showing a configuration of antenna 22 in accordance with a loop circuit, such as the one depicted in FIG. 5A. Antenna 22 is connected to control unit 20, which is connected via connecting cable 21 to handle 19 and probe 18. In this embodiment, control unit 20 includes an impedance matching device 25. Also in this embodiment several series capacitors 27 are used which are electrically equivalent to the single capacitance C in the electrical schematic diagram in FIG. 5A, but which accomplish tuning of the loop in a more efficient manner than would a single capacitor.

As shown in FIG. 6, patient 100 lies on a patient table 102 which is positioned inside a bore 15 of MRI scanner 12. MRI scanner 12 includes a static magnetic field coil 106, a radiofrequency coil 108 positioned within static magnetic field coil 106. Bore 15 is usually an insulating tube covering both static magnetic field coil 106 and radiofrequency coil 108. Bore 15 is configured such that it does not block fields emitted from magnetic field coil 106 and from radiofrequency coil 108. With patient 100 lying inside MRI scanner 12, RF ablation device 16 may be used to treat a lesion within patient 100. Device includes antenna 22, which in this embodiment is a loop circuit 32, configured to receive RF energy from radiofrequency coil 108. Antenna 22 is electronically connected to control unit 20, which in this embodiment is an impedance matching device 25. In some embodiments, impedance matching may be accomplished in other ways. For example, in some instances, the loop impedance may match the cable impedance with only series capacitors. In other cases, at least a parallel capacitor is used to accomplish impedance matching. Connecting cable 21 connects control unit 20 to probe 18 via handle 19. Probe 18 receives RF energy from control unit 20. Probe 18 is inserted through the skin of patient 100 at an entry point 104, which may be determined via images generated by MRI scanner 12. Probe 18 is then configured to administer heat treatment to the lesion.

### Faraday's Law of Motional Induction

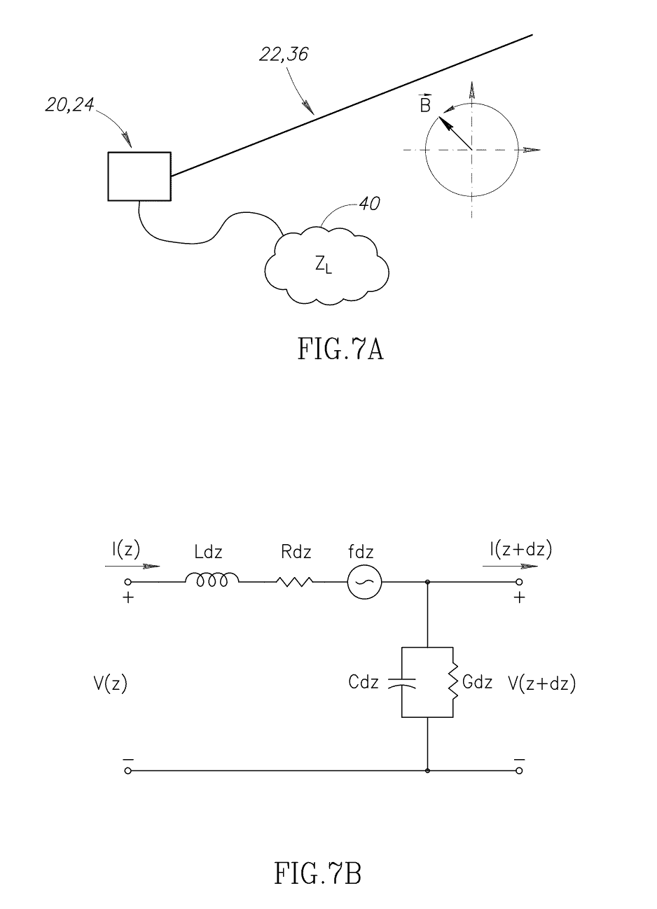

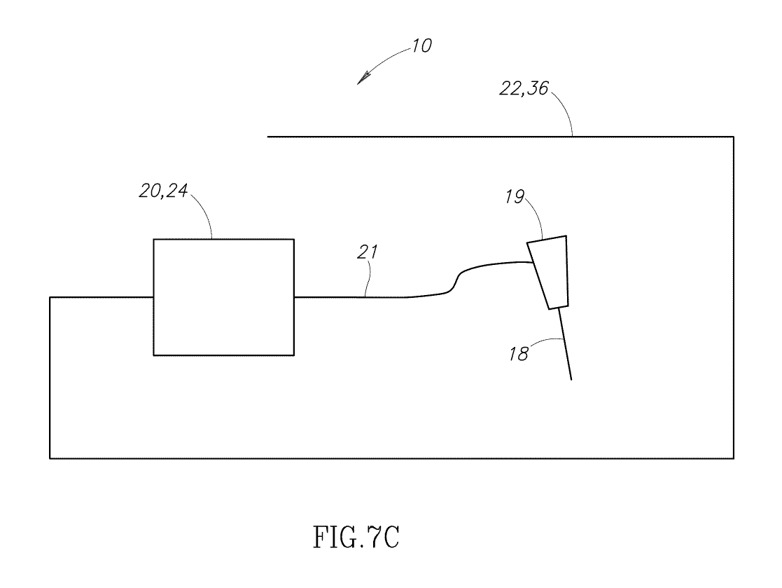

Faraday's law of motional induction states that a moving wire within a static magnetic flux generates a motional EMF. The reverse is also true when rotating magnetic flux from the magnetic coil 106 cuts across an antenna 22 configured as a stationary wire 36 as illustrated in FIG. 7A. The length of the wire 36 needs to be sufficiently long and placed appropriately within magnet bore 15 such that the “pickup” part of it captures an adequate EMF within the magnet bore 15, while the connecting part reaches the tissue 40 to be treated. A control unit 20 may include a tuning circuit 24, used to adjust the effective electrical length of wire 36. Tuning circuit 24 can be as simple as a series capacitor, although other embodiments which do not include a capacitor or which include multiple electrical elements are within the scope of the invention. Since the length of wire 36 is on the order of the wavelength of the operating frequency (e.g., 64 MHz for a 1.5 T static magnetic field), wire 36 acts like a transmission line with standing waves. Even if the two ends of the transmission line are not connected to anything, there is still current in the line. If one end of wire 36 is immersed in tissue, dielectric and/or ohmic heating occurs within the tissue 40 at the tip of wire 36 due to the induced RF voltage in the line. To illustrate a simple mathematical analysis without the use of control unit 20, assume the straight (“pickup”) portion of wire 36 is placed parallel to the magnet axis at radius r from the center of the magnet bore 15, the other end of wire 36 is in contact with tissue 40, and the transmission line electrical network model shown in FIG. 7B is applicable. In FIG. 7B L, R, C and G are respectively the inductance, resistance, capacitance and conductance per unit length of the transmission line. I and V are respectively the current in and voltage across the transmission line at position z along the line. The total length of the line is l. Then the induced distributed EMF f is given by

f=rωB,a<z<b (3)

where magnetic flux cuts the wire only from point a to point b along the line. With this model, the current and the voltage on the line satisfy the inhomogeneous Helmholtz equation which can be solved by the Green's function method, resulting in

$$\begin{matrix}I ( z ) = ( j ω C + G ) {\int }_{0}^{l} g ( z , {z}^{′} ) f ( {z}^{′} ) d {z}^{′} & ( 4 ) \\ V ( z ) = - {\int }_{0}^{l} \frac{d g ( z , {z}^{′} )}{d z} f ( {z}^{′} ) d {z}^{′} & ( 5 )\end{matrix}$$

where g(z,z′) is the Green's function that satisfies the inhomogeneous Helmholtz equation. With the boundary conditions

I(0)=0 (6)

V(l)/I(l)=Z_{L} (7)

the Green's function solution is:

$$g ( z , {z}^{′} ) = \frac{1}{2 γ ( {e}^{γ l} + {Γ}_{L} {e}^{- γ l} )} \times \{ \begin{matrix}( {e}^{- γ z} - {e}^{γ z} ) ( {e}^{- γ ( {z}^{′} - l )} - {Γ}_{L} {e}^{γ ( {z}^{′} - l )} ) , z < {z}^{′} \\ ( {e}^{- γ {z}^{′}} - {e}^{γ {z}^{′}} ) ( {e}^{- γ ( z - l )} - {Γ}_{L} {e}^{γ ( z - l )} ) , z > {z}^{′}\end{matrix} \qquad ( 8 )$$

where γ is the complex propagation constant. Using this solution, the current at the tip of the line can be approximated as

$${I}_{L} \approx \frac{ω r B ( {Γ}_{L} - 1 )}{γ Z ( {e}^{γ l} + {Γ}_{L} {e}^{- γ l} )} ( cosh ( γ b ) - cosh ( γ a ) ) \qquad ( 9 )$$

where Z is the characteristic impedance of the line.

It should be noted that the pickup portion of wire 36 is not required to be straight, and that curved and other wire configurations are all within the scope of the invention. For a wire 36 with arbitrary shape, the induced EMF at any point along the wire depends on the appropriate vector components of the RF field with respect to the wire direction at that point.

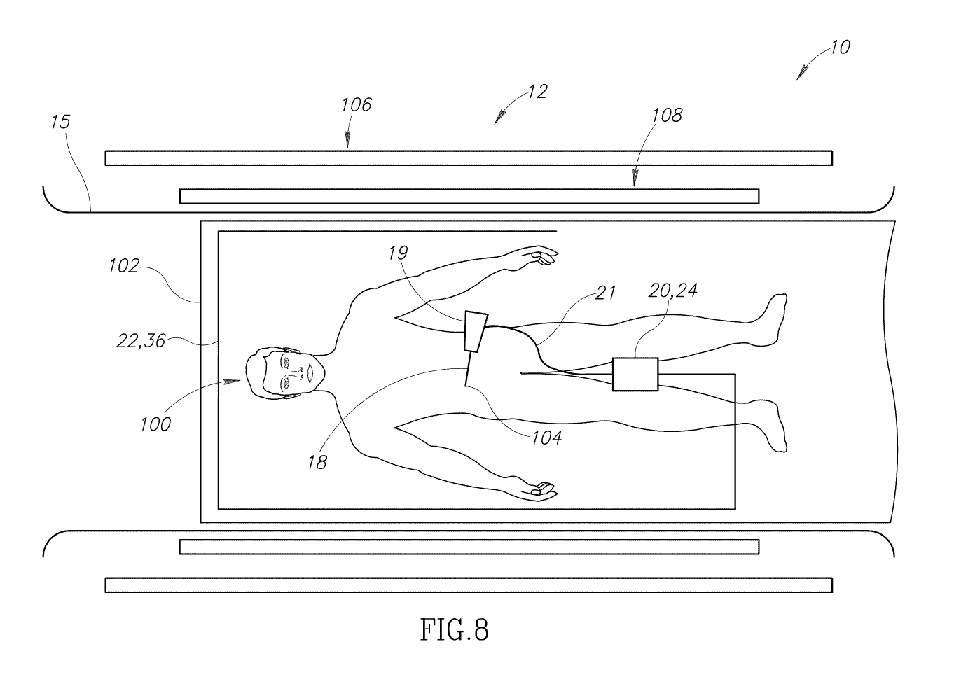

Reference is now made to FIGS. 7C and 8, which are schematic illustrations showing a configuration of antenna 22 in accordance with the circuit diagram of FIG. 7A. In this embodiment, antenna 22 is a wire 36. Antenna 22 is connected to control unit 20, which is connected via connecting cable 21 to handle 19 and probe 18. In this embodiment, control unit 20 includes a tuning circuit 24.

As shown in FIG. 8, patient 100 lies on a patient table 102 which is positioned inside a bore 15 of MRI scanner 12. MRI scanner 12 includes a static magnetic field coil 106, a radiofrequency coil 108 positioned within static magnetic field coil 106. The bore 15 is usually an insulating tube covering both static magnetic field coil 106 and radiofrequency coil 108. Bore 15 is configured such that it does not block fields emitted from magnetic field coil 106 and from radiofrequency coil 108. With patient 100 lying inside MRI scanner 12, RF ablation device 16 may be used to treat a lesion within patient 100. Device includes antenna 22, which in this embodiment is a wire configuration 36, configured to receive RF energy from radiofrequency coil 108. Antenna 22 is electrically connected to control unit 20, which in this embodiment is a tuning circuit 24. In another embodiment, the tuning circuit 24 might not be used if the RF energy picked up by antenna 22 is adequate for heating the tissue without further tuning. In yet another embodiment, the control unit 20 might include one or more thermocouple processor 28 and heat controller 26 components as shown in FIG. 3. Connecting cable 21 connects control unit 20 to probe 18 via handle 19. Probe 18 receives RF energy from control unit 20. Probe 18 is inserted through the skin of patient 100 at an entry point 104, which may be determined via images generated by MRI scanner 12. Probe 18 is then configured to administer heat treatment to the lesion.

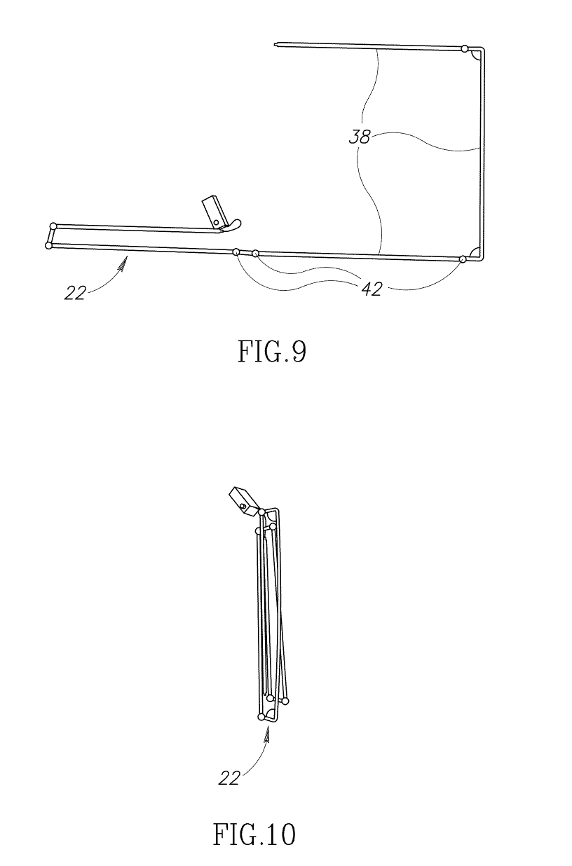

Reference is now made to FIGS. 9 and 10, which are schematic illustrations of antenna 22 having a configuration of wire 36, in accordance with embodiments of the present invention. Wire 36 includes rods 38 and joints 42, such that antenna 22 may be expanded, as in FIG. 9 or folded into a smaller configuration, as in FIG. 10. Other embodiments of antenna 22 include wire 36 affixed to the patient table 102 or other locations within MRI scanner 12 substantially within the RF field of RF coil 108. Antenna 22 may be a disposable device or a nondisposable device.

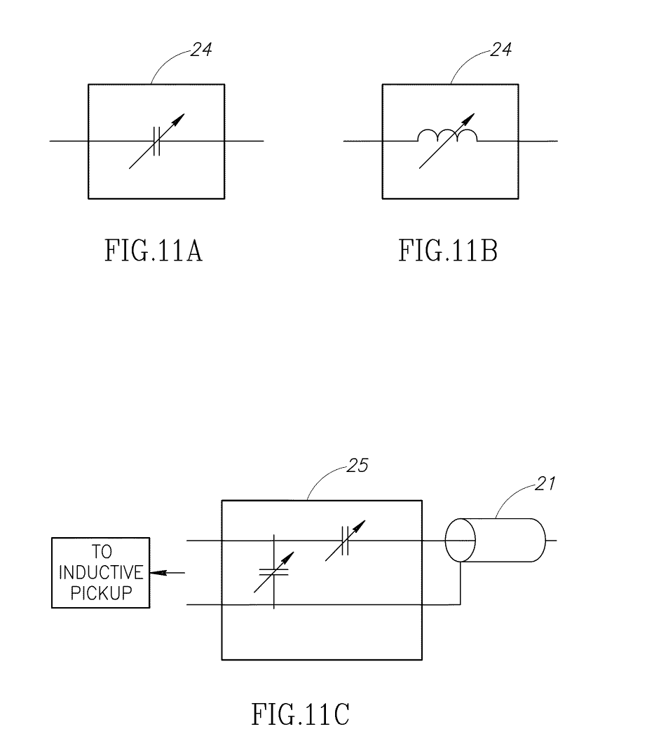

Reference is now made to FIGS. 11A, 11B and 11C, which are schematic illustrations of tuning circuits 24 (FIGS. 11A and 11B) and impedance matching devices 25 (FIG. 11C), in accordance with embodiments of the present invention. In one embodiment, as shown in FIG. 11A, tuning circuit 24 includes a single series capacitor, the capacitance of which may be adjustable. In another embodiment, as shown in FIG. 11B, tuning circuit 24 includes an inductor, the inductance of which may be adjustable. Any suitable combination of adjustable or fixed capacitances and/or inductances and/or electronic elements which accomplish tuning of wire 36 is within the scope of the invention. In one embodiment, as shown in FIG. 11C, impedance matching device 25 includes a pair of adjustable capacitors connected in a series/parallel network. Any suitable network of fixed or adjustable electronic elements which are connected to accomplish impedance matching of the inductive pickup to the connecting cable 21 is within the scope of the invention.

### EXAMPLES

### I. Simulations

In order to better understand the safety and performance issues related to the proposed invention, we simulated the operation of an MR-driven RFA device in the body RF coil of a 1.5 T MRI scanner. The simulation was carried out using the Remcom, Inc. (State College, Pa., USA) XFDTD 7.0 (XF7) 3D electromagnetic simulation software package, which is based on the FDTD (finite difference time domain) method. The modeled body coil had dimensions of 60 cm long and 60 cm diameter, and was a 16 rung highpass birdcage coil. It was first tuned to 1.5 T so that the field within the center of the body coil was homogenous. Then, its performance with a rectangular solid (box phantom) 7 cm tall, 31 cm long, 23 cm wide, with the electrical properties of liver tissue (dielectric constant 70.62, conductivity 0.55 S/m) placed at the isocenter was recorded as a reference. Finally, the RFA device, modeled as a simple wire which captures RF energy from the body coil by electromagnetic induction, was placed in the model geometry with its tip embedded in the box phantom corresponding to our experiments. The RFA device was modeled as PEC (perfect electrical conductor) material. The simulation grid (spatial resolution) was chosen to be 1 cm.

The unloaded simulated birdcage coil was tuned to 64.178 MHz using 40 pF capacitors on the end rings (S₁₁=−23 dB). The calculated |B₁⁺| field contour map was fairly homogeneous. By comparing the |B₁⁺| fields within the simulated phantom without and with the RFA device, it was found that the body coil was highly coupled to the RFA device due to magnetic flux density cutting through the device. This agrees with experimental results (below) which show in the images a significant brightening artifact at the location of the device (most visibly at its tip) which aids in its visualization. In contrast to other inventions, the present invention includes this extremely useful characteristic of providing position information when imaging is performed, while not being physically connected to the scanner, and without the need for reception of a separate signal (e.g., from a surface RF coil or catheter RF coil). The reflection coefficient S₁₁ of the body coil changed from −16.2 dB with the simulated phantom only to −3.6 dB with the RFA device also present. Thus it is expected that the overall field intensity averaged over the entire body RF coil volume would be lower when the device is present, possibly affecting the operation of the scanner or causing over-estimation of specific absorption rate (SAR, a measure of the RF heating effect on tissue in the MRI scanner). However, in experiments the scanner always performed normally and scanning was never interrupted by excessive reflected power. The calculated ratio of the average simulated SAR (based on a 1 g average) of the phantom with and without the RFA device was 0.21, indicating that the overall field was lower when the RFA device was present. However, the ratio of the maximum local SAR of the box phantom in the vicinity of the wire tip was 2.65. The maximum local SAR occurs at the tip of the RFA device, demonstrating that the device has a significant energy localization effect exactly as desired.

### II. Experiments

Experiments were carried out in a Siemens (Erlangen, Germany) Avanto 1.5 T scanner with a Larmor frequency of 63.64 MHz. The scanner contains a 57 cm long body coil with diameter 61 cm. For the transformer induction experiments, a pickup loop circuit was built using 5 mm adhesive copper tape on ABS sheet and high voltage nonmagnetic ceramic multilayer capacitors (American Technical Ceramics, Huntington Station, N.Y., USA). The circuit was tuned to resonance at the scanner operating frequency by checking the transmission between two magnetic pickup loops overlapped so as to have minimum mutual inductance when far from a resonant circuit. A BNC jack was inserted into the loop in series so that a nonmagnetic Teflon dielectric 50 ohm coaxial cable (part number 50HCX-15, Temp-Flex Cable, South Grafton, Mass., USA) could be connected to it. A nonmagnetic high voltage ceramic variable capacitor (part number SGNMNC3708E, Sprague-Goodman Electronics, Westbury, N.Y., USA) in series with the coaxial cable permitted the cable to be adjusted to quarter wavelength. The pickup loop circuit was placed on the patient table of the scanner and positioned near the magnet isocenter.

The end of the coaxial cable was placed into a phantom consisting of a polyethylene tub of normal saline gel made with 1% (by weight) agar to simulate tissue. The gel also contained nickel sulfate to reduce the T₁ relaxation time. The input impedance of the coaxial cable when in contact with the gel was 52−j32Ω at 63.64 MHz. For all experiments, the phantom was placed next to the loop on the patient table.

A Neoptix (Quebec, Canada) T1 fiber optic temperature probe was attached 5 mm behind the tip of the cable using heat shrink tube. The probe was connected to the Neoptix Reflex fiber optic thermometer signal conditioner which sent a continuous stream of temperature readings in ASCII format to a laptop computer through a serial port. Since the Neoptix does not provide a time stamp, the time resolution between the temperature points was first measured. A flag was inserted into the captured ASCII stream by sending the “h” character to the signal conditioner (to invoke the help message which was then embedded in the data stream) immediately before and after the heating pulse sequence as a time stamp. The ASCII data was later processed in MATLAB to yield the temperature profile (temperature vs. time data) during the heating scan.

For the motional induction experiments, a 26 gauge Teflon insulated silver plated solid wire (part number 2853/1 WH005, Alpha Wire Company, Elizabeth, N.J., USA) was taped to the patient table of the scanner. A segment of wire continued to the saline agar gel phantom. The fiber optic temperature probe was attached 5 mm behind the wire. The Teflon insulation of the tip of the wire was stripped to expose 5 mm of bare conductor which was then dipped into the saline agar gel phantom.

A high RF duty cycle turbo spin echo (TSE) pulse sequence and a low RF duty cycle gradient echo (GRE) pulse sequence were used for RF excitation. The TSE sequence started with a 90° pulse, which was followed by three 150° pulses spaced by TE=8.43 ms. TR was 643 ms and the total scan duration was 110 s. The GRE used a 1 ms 25° pulse, with TR=337 ms for a total scan duration of 64 seconds.

Additional experiments were conducted with bovine liver sections obtained from the grocery. Similar results were obtained as with the gel phantom, except that readily visible ablation lesions due to irreversible thermal damage were created in the liver tissue. With a bare wire exposure of 5 mm, roughly spherical lesions of diameter 5 mm could be readily created with less than 1 min of heating, and 20 mm of bare wire created cigar-shaped lesions roughly 20 mm long. Because the liver tissue could be coagulated, the thermal profiles often exhibited a maximum temperature well below the maximum 100° C. temperature (the water boiling temperature) achievable with the gel. Cycles of buildup of coagulation (eschar) and breakthrough on the exposed wire would lead to current limiting, then continued heating, followed by more buildup, yielding heating curves with unstable limiting characteristics. To compare the ablation lesions achieved by the MRI procedure with those of conventional RFA, several lesions were produced in a liver specimen with a Valleylab CoolTip RF ablation system that is used in the clinic for tumor treatment. No saline cooling was used. The ablations were conducted by a radiologist who commonly treats tumors with RFA. Lesions of similar size and character to those produced by the MRI procedure were obtained, but typically in somewhat longer times.

The chemical shift of water protons has a well known variation with temperature of about −0.01 ppm/° C., and is the basis for the proton resonance frequency shift (PRFS) method for measuring the tissue temperature. The phase of a GRE image reflects the resonance frequency offset of water protons due to temperature changes. Therefore, brief (3.4 scan duration) single slice phase sensitive GRE images positioned to include the wire tip in the plane of the image were obtained immediately before and after heating pulse sequences. The phase of each image was unwrapped using an adaptation of the Jenkinsen phase unwrapping algorithm [M. Jenkinson, Fast, automated, n-dimensional phase-unwrapping algorithm. Magn Reson Med 49: 193-197 (2003)]. In our adaptation of the Jenkinsen method, regions of the image above a certain signal intensity threshold are segmented depending on the range of pixel phase values, segmenting the image pixels into spatial clusters. Spatially adjacent clusters are compared and conditionally combined depending on their relative phase, and whether wrapping around 180° is required. The process is repeated until only a single cluster remains. The phase difference in each pixel between the before-heating and after-heating images is scaled to yield the temperature change.

### Results and Discussion

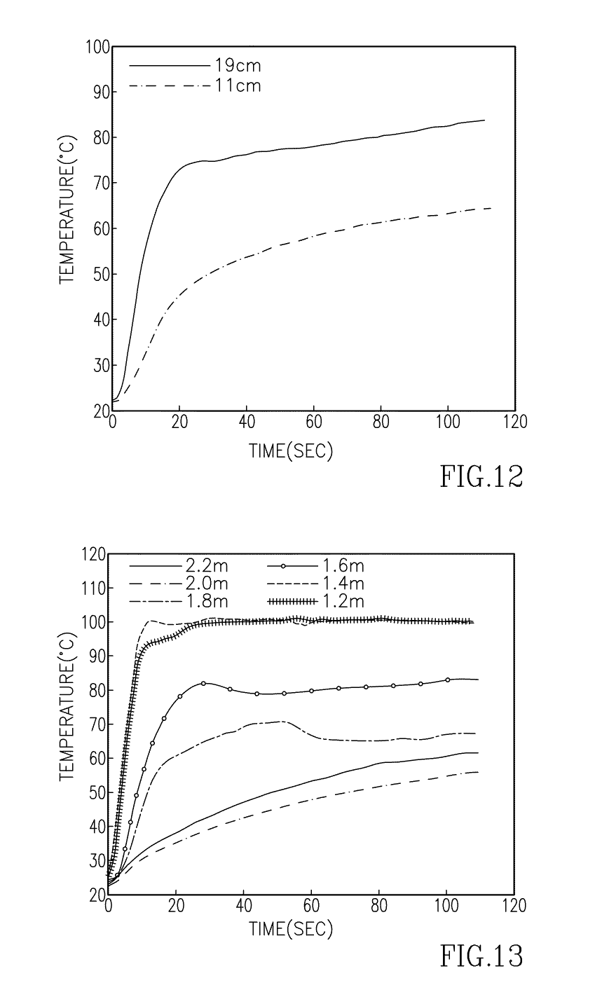

Reference is now made to FIG. 12, which is a graphical illustration showing the temperature at the tip of the coaxial cable within the agar as a function of time for the 11 cm and 19 cm square loop circuits using the high RF duty cycle TSE pulse sequence. It was expected from Equation 1 that the larger loop would exhibit a higher heating rate compared to the smaller one, and this was observed. During the pulse sequence, surface currents flowing on the outer conductor of the coaxial cable resulted in some heating of the cable body. The inner conductor touching the agar would oxidize after multiple trials, reducing the heating efficiency, necessitating cutting off the oxidized portion and restripping the insulation. In addition, repeated heating of the gel at the same location appeared to cause some local compositional changes in the gel, because the heating seemed to change over time. This could have resulted from increased gel impedance. The position of the cable in the gel was therefore changed frequently.

The body coil is designed to generate a uniform magnetic flux covering a cylindrical volume of length 50 cm along the longitudinal axis (z). Thus, the wire was taped from z=−25 cm to z=+25 cm (where z=0 cm means isocenter) to the magnet bore. Varying the length of the wire from 2.2 m to 1.2 m, the temperature profile was affected by the wavelength effect. Reference is now made to FIG. 13, which is a graphical illustration showing the temperature measured at the tip of different length wires as a function of heating time. There was a roughly oscillatory variation of heating rate as a function of wire length, with shorter wires generally yielding greater heating, demonstrating the expected resonant transmission line behavior. At 1.8 m, arcing at the wire tip in the gel was observed. High heating or especially arcing damaged the exposed wire surface, altering its contact resistance, yielding heating profiles which were not monotonic. Because of the difficulty in positioning the wire in a reproducible manner as the length was varied, it was not possible to observe a strictly periodic variation in heating rate with length change. The long wire results were therefore not as reproducible as the loop results. In all cases the tip temperature does not exceed 100° C. because the water in the gel boils at this temperature.

The TSE pulse sequence imposes high SAR on the patient, and so we investigated using a lower RF duty cycle GRE pulse sequence. To increase the coupling between the scanner and the wire, the wire was made longer by taping to the right side of the bore, extended across the bore and taped to the left side, in both cases from z=−25 cm to z=+25 cm. A variable capacitor was soldered in series with the wire about 1 m away from the immersed wire tip to adjust the effective electrical length of the wire, providing a more convenient and reversible means to tune the transmission line. By optimizing the capacitance, it was found that 6 pF gave the maximum heating effect for this particular configuration of wire.

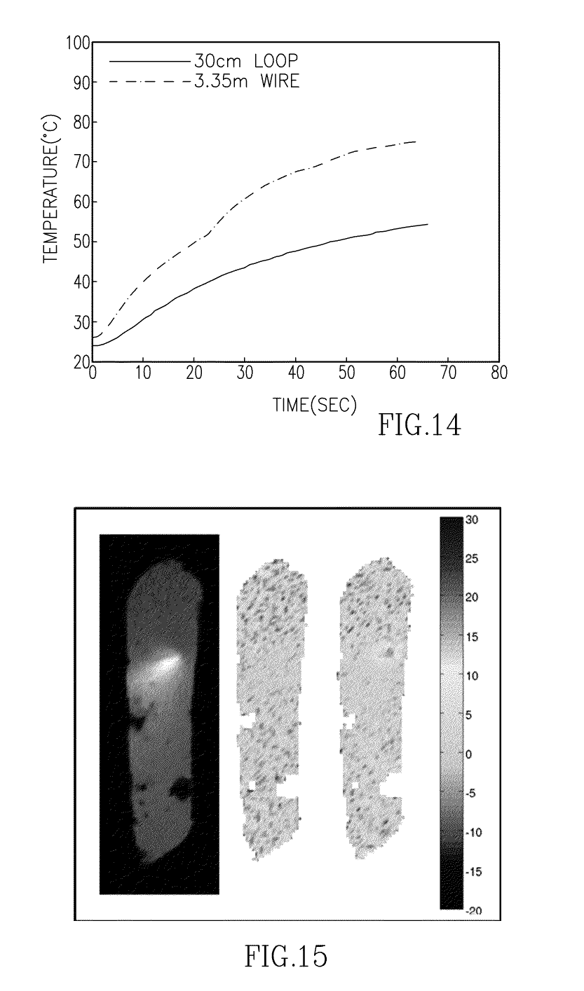

Reference is now made to FIG. 14, which is a graphical illustration showing a comparison of the longer wire with a larger 30 cm square loop. Although both configurations produced effective heating, the wire outperformed the loop because the wire's effective coverage area was larger.

Reference is now made to FIG. 15, which is a graphical illustration showing thermal imaging results in a specimen of bovine liver using a long wire pickup antenna. The magnitude image (on the left of FIG. 15) showing a cross section of the liver specimen into which the wire tip was inserted reveals an artifact (bright spot) due to the wire tip, which helps to visualize the placement of the device in the tissue. The middle and right cross sectional images represent the temperature of the liver tissue measured by appropriate processing of image data from the MRI scanner. The middle image was obtained immediately before RF heating using the ablation device. The right image was obtained immediately after RF heating using the ablation device. The color scale on the extreme right shows the temperature increase over the ambient temperature in the two temperature images. Before heating, the temperature of the tissue is approximately uniform (middle image). Note that the thermal image is free of the RF artifact, even though the wire is present, because the thermal image depends only on the signal phase change, and not the signal magnitude. After RF heating using the ablation device (right image) the tissue hot spot is plainly visible at the location of the wire tip. The temperature increase of 20° C. determined from the right temperature image agrees well with the 22° C. temperature rise reported by the fiber optic thermometer.

In vivo, blood circulation and perfusion is highly effective at removing heat deposited by the RF applicator, and reduces the heating efficiency considerably. In addition, the overall SAR to the patient is limited by U.S. Food and Drug Administration guidelines, requiring the RF applicator to be highly efficient so that relatively low SAR pulse sequences can be used. These considerations will be important when the invention is used clinically, but are not relevant to demonstrating the principles of the invention.

Both equations 1 and 9 show that using higher field scanners (which operate at higher RF frequencies) should increase the efficiency of these RFA devices. Because signal-to-noise ratio and image quality generally increase with field, performing RFA treatment at higher field should lead to shorter treatment times and better real time treatment monitoring. Some experiments were performed at a scanner static magnetic field strength of 3.0 T (RF frequency 123 MHz), demonstrating both the expected higher levels of heating, and the expected shorter wavelength transmission line effects. The use of all scanner magnetic field strengths, and the use of the invention outside of an MRI scanner but employing the above described electromagnetic induction effects to heat a needle tip are all within the scope of the invention.

By dispensing with a separate RF generator and external connecting cables, tuned loops or long wires within the scanner offer alternatives for sources of RF energy to perform ablations. The generation of an EMF to drive RF current in these devices can be described with Faraday's law of induction, based on an analogy between the rotating RF field of the body coil with the rotating coils of an electric power generator. Experiments show that sufficient heat energy can be extracted from the RF field of the scanner using typical clinical pulse sequences to meet the requirements of RF ablation. The pulse sequence RF duty cycle can be used to control the rate of heat production. Because the ablation is carried out in the MRI scanner, real time guidance is possible, and tissue temperature, perfusion, coagulation and other parameters are readily imaged. In particular, the ability to measure tissue temperature during the procedure should result in better outcomes because the temperature of the tumor margins can be directly measured. The elimination of the ground pad and other external wired connections eliminates some of the hazards of conventional RFA.

While certain features of the present invention have been illustrated and described herein, many modifications, substitutions, changes, and equivalents may occur to those of ordinary skill in the art. It is, therefore, to be understood that the appended claims are intended to cover all such modifications and changes as fall within the true spirit of the present invention.

What is claimed is:

1. A wireless heat ablation device for use inside a bore of a magnetic resonance imaging scanner, the device comprising:
an antenna configured to wirelessly receive RF energy from the magnetic resonance imaging scanner; and
a probe having an electrically conductive tip, said probe electronically connected to said antenna, and configured to receive said RF energy, said probe further configured to be positioned within tissue and to provide heat to the tissue by said RF energy.

2. The device of claim 1, further comprising a control unit in electrical communication with said antenna, said control unit configured for receiving said RF energy from said antenna and for coupling said received RF energy, and for sending the coupled RF energy to said probe.

3. The device of claim 1, wherein said antenna comprises a loop circuit.

4. The device of claim 3, wherein said antenna has dimensions of 10-50 cm in length and width.

5. The device of claim 2, wherein said control unit comprises an impedance matching device.

6. The device of claim 1, wherein said antenna comprises a wire.

7. The device of claim 6, wherein said antenna has a length of 20-80% of a wavelength of the RF signal.

8. The device of claim 6, wherein said wire comprises rods and joints such that said wire may be folded or expanded.

9. The device of claim 2, wherein said control unit comprises a tuning circuit.

10. The device of claim 9, wherein said tuning circuit comprises at least one of: a variable capacitor and a variable inductor.

11. The device of claim 9, wherein said tuning circuit is configured to vary a length of the wire.

12. The device of claim 1, wherein said electrically conductive tip is an un-insulated distal portion of a length of an insulated electrically conductive needle, hollow tube, or catheter.

13. A method of heat ablation for use inside the bore of a magnetic resonance imaging scanner, the method comprising:
positioning a wireless heat ablation device inside the bore of said magnetic resonance imaging scanner, the magnetic resonance imaging scanner providing RF transmission at a frequency;
tuning said wireless heat ablation device to approximately the frequency of the RF transmission of said scanner;
receiving RF energy in said wireless heat ablation device based on said RF transmission;
providing heat to a tip of said wireless heat ablation device based on said received energy;
determining a treatment location for said heat ablation device by imaging a treatment area using said magnetic resonance imaging scanner; and
heating said treatment location using said heated tip of said wireless heat ablation device.

14. The method of claim 13, wherein said step of providing heat may be controlled by changing said tuning of said wireless heat ablation device.

15. The method of claim 13, wherein said step of providing heat may be controlled by changing the average power of radiofrequency transmission of said magnetic resonance imaging scanner.

16. The method of claim 12, wherein said step of providing heat may be controlled by changing the average power of radiofrequency transmission of said magnetic resonance imaging scanner and by changing said tuning of said wireless heat ablation device.
